(19) United States
(12) Patent Application Publication
(10) Pub. No.: US 2023/0016297 A1
(43) Pub. Date: Jan. 19, 2023

(54) DEVICES, SYSTEMS, AND METHODS FOR ORTHOPEDICS

(71) Applicant: BULLSEYE HIP REPLACEMENT, LLC, Las Vegas, NV (US)

(72) Inventor: Michael GILLMAN, Laguna Beach, CA (US)

(73) Assignee: BULLSEYE HIP REPLACEMENT, LLC, Las Vegas, NV (US)

(21) Appl. No.: 17/864,656

(22) Filed: Jul. 14, 2022

Related U.S. Application Data
(60) Provisional application No. 63/362,378, filed on Apr. 1, 2022.
(60) Provisional application No. 63/269,687, filed on Mar. 21, 2022.
(60) Provisional application No. 63/203,280, filed on Jul. 15, 2021.

(51) Int. Cl. A61B 34/10; G16H 20/40

(52) U.S. Cl. CPC A61B 34/10; G16H 20/40; A61B 2034/104; A61B 2034/105

(57) ABSTRACT

A surgical planning and evaluation method may include receiving an image of a patient's anatomy before a surgery and generating a surgical plan for performing the surgery. The surgical plan may include a planned surgical result. The method may include receiving an image of the patient's anatomy after surgery. The image may include data representing the achieved surgical result. The method may include digitally comparing the planned surgical result with the achieved surgical result and generating a quantification the surgical result based on the digital comparison.

### CROSS-REFERENCE

This application claims the benefit under 35 U.S.C. § 119(e) of U.S. Provisional Patent Application No. 63/203,280, filed Jul. 15, 2021, of U.S. Provisional Patent Application No. 63/269,687, filed Mar. 21, 2022, and of U.S. Provisional Patent Application No. 63/362,378, filed Apr. 1, 2022, the disclosures of which are incorporated, in their entirety, by this reference.

### BACKGROUND OF THE INVENTION

### Technical Field

The present disclosure relates to systems, devices, and methods for postoperative verification of implant placement, such as hip, knee, and spinal implants, and bone fracture repair, and more particularly, to post-operative quantification implant placement and bone fracture reduction based on preoperative planning and postoperative imaging.

### Description of the Related Art

One method of repairing fractured bones is to reduce the bone fracture and then fix the fractured bone in a reduced position. Improper reduction and fixing may lead to pain and reduced movement or use of extremities or other parts of the body. An orthopedic surgeon performing a fracture repair procedure seeks to ensure, through surgery, accurate reduction and fixation of the bone through proper reduction of the bone and placement of implants to fix the reduced bone. However, current methods of evaluating fracture reduction are either non-existent or less than ideal in their evaluation of surgical success.

One method of treating hip and other joints with arthritis and other medical conditions is to replace surfaces of articulating joints with prosthetic devices through surgical procedures. The prosthetic devices should be accurately designed and manufactured, and installed correctly in order to relieve pain and provide an effective treatment method for such ailments. An orthopedic surgeon performing such joint replacement on a patient seeks to ensure, through surgery, adequate placement of the prosthetic and proper reconstruction of the joint being replaced.

One method of treating spinal conditions is via surgery, such as spinal decompression, spinal fusion, spinal disc replacement, and spinal deformity correction. Current methods do not accurately plan prior to surgery or evaluate surgical results after surgery. Current methods of evaluating prosthetic spinal surgical success are either non-existent or less than ideal in their evaluation of surgical success as they do not plan prior to surgeries or evaluate post surgical success.

Current methods of fracture reduction and implant placement, such as prosthetic placement, do not provide for accurate postoperative qualitative and quantitative analysis and comparison with preoperative predictions. For example, many postoperative evaluations merely include external assessment though manual probing or articulation of the bones and joints. Such assessments do not compare the preoperative placement and reduction goals with the actual postoperative placement and reduction achieved.

### SUMMARY OF THE INVENTION

With the assistance of computer-generated data derived from preoperative CT, MM, or other scans, such as X-rays, surgeons can more effectively determine proper positions of properly placed implants and properly reduced bones in a patient through 3-D modeling and rendering. Through postoperative CT, MM, or other scans, such as X-rays, the desired or preoperative placement or reduction may be compared to the achieved placement or reduction. Such comparisons may aid in training surgeons and improving their skills, aid in evaluating surgeons for use by patients in selecting a doctor, allow for surgeons to promote their skills and outcomes, and for evaluation by hospitals and insurance companies for hiring and pay or reimbursement.

The devices, systems, and the methods described herein aid in the quantification of the differences between the planned surgical result and the achieved surgical result. Feedback based on the quantification of the differences between the planned surgical result in the achieved surgical result may be provided to the surgeon or other parties to the surgery for teaching or evaluation of the surgeons. The feedback may also be used by insurers or other third-parties for use in providing surgical achievement ratings that insurers, potential patients, and others may use in order to choose a surgeon. In some embodiments, reimbursement of medical expenses by insurance agencies may be determined based, in part, on the quantification of the differences between the planned surgical result and the achieved surgical result.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a method according to one or more embodiments disclosed herein;

FIG. 2 depicts a system according to one or more embodiments disclosed herein;

FIGS. 3A and 3B depict a front view of a left and right humerus bones, respectively, according to one or more embodiments disclosed herein;

FIG. 4A depicts a front view of a preoperative plan for reducing a humerus fracture and an installed specific jig according to one or more embodiments disclosed herein;

FIG. 4B shows a top view of the patient specific jig of FIG. 7A;

FIG. 5 depicts a front view of an image of a humerus with a reduced fracture and an installed implant of a preoperative plan according to one or more embodiments disclosed herein;

FIG. 6 depicts a front view of an image of a humerus with a postoperative reduced fracture and an installed implant according to one or more embodiments disclosed herein;

FIG. 7 depicts a front view of an image a pelvis with a postoperative installed implant according to one or more embodiments disclosed herein;

FIG. 8 depicts a method according to one or more embodiments disclosed herein;

FIGS. 9 and 9B depict aspects of an anterior cruciate ligament (ACL) reconstruction according to one or more embodiments disclosed herein; and

FIG. 10 depicts a rotor cuff repair according to one or more embodiments disclosed herein.

### DETAILED DESCRIPTION OF THE INVENTION

The present disclosure pertains to the use of computer-generated data derived from preoperative CT, MM, or other scans, such as X-rays, surgeons to preoperative determine proper positions of properly placed implants and properly reduced bones in a patient through 3-D modeling and rendering and using postoperative CT, MM, or other scans, such as X-rays, the desired or preoperative placement or reduction may be compared to the achieved placement or reduction.

In preparation for orthopedic surgery, a variety of diagnostic images of the patient may be obtained utilizing CT, MM, and other scans, such as x-rays, to generate three-dimensional (3-D) models of the patient's bone structure. From such 3-D models, the surgeon may determine the amount of bone to be removed, such as during a resection procedure, the extent of a fracture and/or implant installation location and/or the orientation of the implant to be secured to the patient's anatomy during surgery and/or the proper size of the implant.

The methods and systems disclosed herein are based at least in part on pre-operating (preoperative) imaging and at least in part on orthopedic surgical procedures based upon the preoperative methods and systems. Preoperative imaging has a number of different purposes and generally is performed to help guide the surgeon during the surgical procedure. Preoperative imaging may allow for patient-specific tools or implants to be formed, etc. The present disclosure may be part of a system for designing and constructing one or more patient-specific jigs for use in an orthopedic surgical procedure in which a fixation implant is prepared, oriented, and implanted. In some embodiments, the present disclosure may be part of a system for designing and constructing one or more patient specific jigs resecting a patient's anatomy, implanting an implant, and confirming that implant is properly located, such as during hip replacement, and other joint replacement, fracture surgery or spinal implant surgery, or spinal implant surgery.

The referenced systems and methods are now described with reference to the accompanying drawings, in which one or more illustrated embodiments or arrangements of the systems and methods are shown in accordance with one or more embodiments disclosed herein. Aspects of the present systems and methods can take the form of an entirely hardware embodiment, an entirely software embodiment (including firmware, resident software, micro-code, etc.), or an embodiment combining software and hardware. One of skill in the art can appreciate that a software process can be transformed into an equivalent hardware structure, and a hardware structure can itself be transformed into an equivalent software process. Thus, the selection of a hardware implementation versus a software implementation is one of design choice, and is left to the implementer.

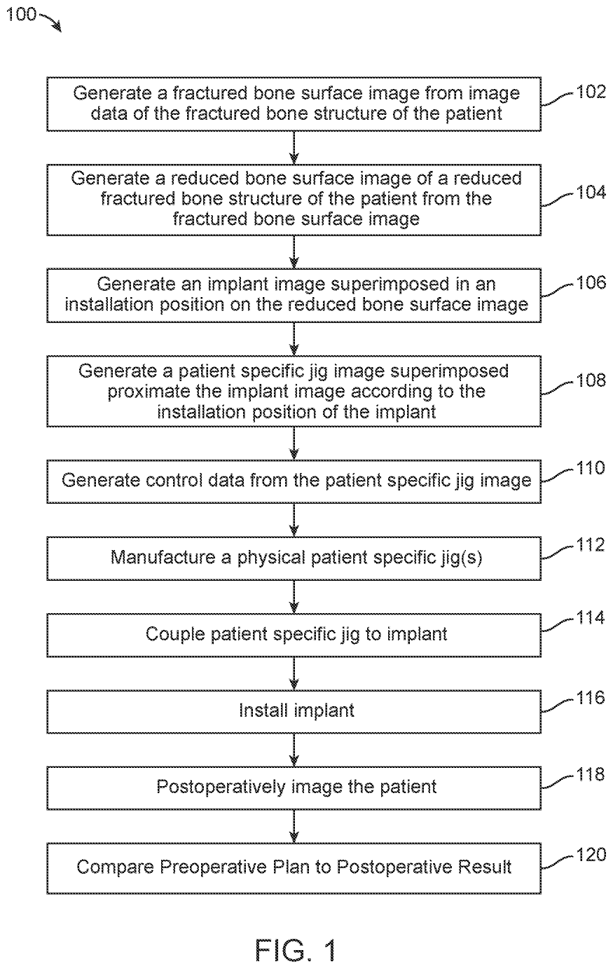

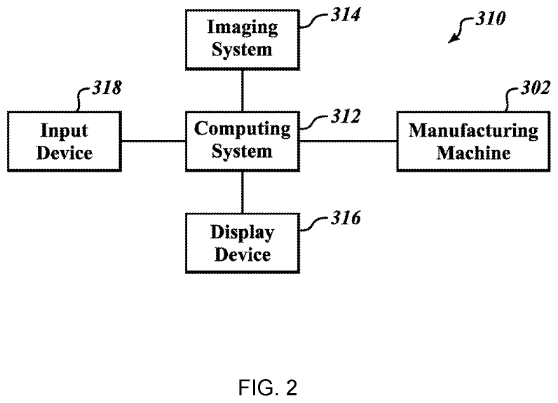

FIG. 1 shows a flow diagram illustrating a method pertaining to preoperative imaging and planning according to aspects of the present disclosure. FIG. 2 shows a system for carrying out the methods of the present disclosure, such as that described with reference to FIG. 1. FIG. 2 shows a simplified system 310 of devices that may be used to carry out the methods of the present disclosure. The system 310 comprises a computing system 312 coupled to an imaging system 314. The imaging system 314 captures patient image data and transfers the data to the computing system 312. The computing system 312 processes such data and transmits the data to a display device 316 for display of images and other data. An input device 318 receives input from a computer or an operator (such as a surgeon) and transmits inputted information to the computing system 312 for processing. Such input devices 318 are well known in the art and will not be described in greater detail. The imaging system 314 may include a bone imaging machine for forming three-dimensional image data from a bone structure of a patient. The computing system 312 may include a patient-specific device generator for processing and generating images, and a patient-specific device converter for generating design control data. A manufacturing machine 320 receives the control data from the computing system 312 for making devices described herein, including patient-specific jigs.

The computing system 312 may be a data processing system that may be used in executing any of the methods and processes described herein. The computing system 312 typically includes at least one processor that communicates with one or more peripheral devices via bus subsystem. These peripheral devices typically include a storage subsystem, including a memory subsystem and file storage subsystem, a set of user interface input and output devices, and an interface to outside networks. This interface may be coupled to corresponding interface devices in other data processing systems via a communication network interface. Data The computing system 312 can include, for example, one or more computers, such as a personal computer, workstation, mainframe, laptop, and the like.

The input devices 318 are not limited to any particular device, and can typically include, for example, a keyboard, pointing device, mouse, scanner, interactive displays, touchpad, joysticks, etc. Similarly, various user interface output devices can be employed in a system of the invention, and can include, for example, one or more of a printer, system/subsystem, controller, projection device, audio output, and the like.

The storage subsystem maintains the basic required programming, including non-transitory computer readable media having instructions (e.g., operating instructions, algorithms, programs, etc.), and data constructs. The program modules discussed herein and instructions for carrying out the methods described herein are typically stored in storage subsystem. Storage subsystem typically includes memory subsystem and file storage subsystem. Memory subsystem typically includes a number of memories (e.g., RAM, ROM, etc.) including computer readable memory for storage of fixed instructions, instructions and data during program execution, basic input/output system, etc. File storage subsystem provides persistent (non-volatile) storage for program and data files and can include one or more removable or fixed drives or media, hard disk, floppy disk, CD-ROM, DVD, optical drives, and the like. One or more of the storage systems, drives, etc. may be located at a remote location, such coupled via a server on a network or via the internet/World Wide Web. In this context, the term “bus subsystem” is used generically so as to include any mechanism for letting the various components and subsystems communicate with each other as intended and can include a variety of suitable components/systems that would be known or recognized as suitable for use therein. It will be recognized that various components of the system can be, but need not necessarily be at the same physical location, but could be connected via various local-area or wide-area network media, transmission systems, etc.

Imaging system 314 includes any means for obtaining a digital representation (e.g., images, surface topography data, etc.) of a patient's anatomy and includes means of providing the digital representation to computing system for further processing. Imaging system 314 may be located at a location remote with respect to other components of the system and can communicate image data and/or information to computing system 312, for example, via a network interface. Manufacturing machine 302 fabricates implants based on preoperative planning. Manufacturing machine 302 can, for example, be located at a remote location and receive data set information from data imaging system 314 via a network interface.

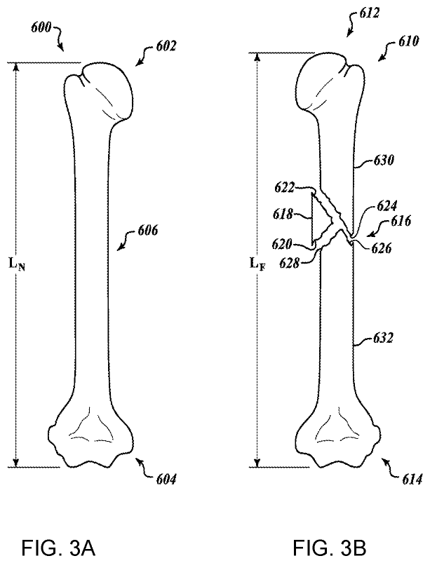

In FIG. 1, a method 100 according to an embodiment may start at block 102. While described with respect to fracture reduction, a similar process may be used for other orthopedic surgeries, such as hip or knee replacement, spinal surgery, etc. At block 102, a bone imaging machine generates a bone surface image from three-dimensional image data from the bone structure of a patient, for example, as shown in FIGS. 3A, 3B, 5, and 6. At block 104, a reduced bone surface image of a reduced fractured bone structure of the patient is generated from the fractured bone surface image, for example, as shown in FIG. 3B. In some embodiments, the three-dimensional image data of the bone structure of a patient may include three-dimensional models of one or more portions of the fractured bone of the patient. In some embodiments, the individual three-dimensional models of the portions of the fractured bone are manipulated and placed in a reduced configuration such that the reduced bone surface image represents a pre-operatively planned reduction of the fractured bone. The pre-operatively planned reduction represents that planned actual reduction of the patient's actual fractured bone. In some embodiments, the image may be of an acetabular cup, pelvis, knee, spine or other bone. In some embodiments, the image may be manipulated to generate preoperatively planned resection models that depict the patient's anatomy after resection or other material removal procedures.

In some embodiments, bone surface images may be made of a patient's corresponding unfractured or healthy bone to aid in determining the reduced bone surface image of the reduced fractured bone structure. For example, in some embodiments, such as that shown in FIGS. 3A and 3B, a right tibia may be fractured, while a left tibia is unfractured. In such embodiments, imaging, such as an x-ray image, may be used to determine the length LN of the left tibia. The lengths of a person's left and right bones, in the embodiments, the left tibia and right tibia, are known to be similar. Thus, the reduced bone surface image of the right tibia may be formed such that the length LF of the reduced fractured right bone corresponds to the length LN of the unfractured left tibia. By corresponding, the lengths are the same or similar to each other.

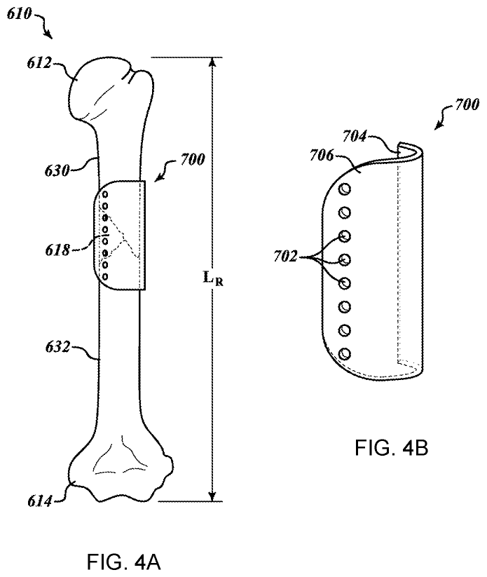

At block 106, a patient-specific device generator generates an implant image superimposed in an installation position on the reduced bone surface image. For example, FIGS. 4A, 5, 6, and 7 include aspects of the preoperative implant image at the installation position. The implant image is positioned in its final, implanted position and orientation, regardless of the state of the patient's bone in the bone surface image. The implant image may be a patient specific implant image, having a surface that is shaped to match or otherwise mate with the bone surface image of the patient. In some embodiments, the implant may be a standard size or non-patient specific implant. A patient specific implant formed based on the implant image may have an actual surface that matches or otherwise mates with the reduced fractured bone of the patient. Similarly, implant images may be generated that depict the preoperative planned position of one or more prosthetics or other implants, such as an acetabular cup, spine fusion implant, knee replacement implants, etc that are positioned with respect to the patient's anatomy in a desired optimal position or range of acceptable positions.

Similarly spinal implant images may be generated that represent the preoperatively planned position of a spine implant, such as those used during decompression, spinal fusion, disc replacement or spinal corrective osteotomy placed. In some embodiments, the implant images may show a range of acceptable positions.

Optionally, at block 108, the patient-specific device generator generates a patient specific jig image superimposed proximate the reduced bone surface image and the implant image according to the installation position of the implant. The patient-specific device generator may use the bone surface image to create a patient-specific device with anatomic engagement members that have an engagement surface that corresponds to, matches, or is the negative contour of the patient's anatomy. In some embodiments, the engagement surface is shaped to nestingly mate with the corresponding surface of the bone of the patient. The patient-specific device generator may use the implant image to generate implant engagement members that engage with features of the implant, such as a surface, end, or aperture of the implant. The patient-specific device generator may also use the implant image and the bone surface image to generate jig alignment features or members.

Optionally, at block 110, a patient-specific device converter generates control data from the patient-specific jig image. The control data may be used by a machine during a manufacturing process to create physical patient-specific jigs by additive or subtractive machining, such as fused deposition modeling, stereolithography, or other methods. At block 112, the manufacturing device creates a physical patient-specific jig.

Optionally, at block 114, the implant may by coupled to the patient specific jig. Coupling the patient specific jig and the implant together may provide for greater ease of handling, transportation, and use as compared to uncoupled implants and jigs.

Optionally, at block 116, a doctor may perform the orthopedic surgery and install an implant, reduce a fracture, or perform other orthopedic procedures as detailed in the preoperative plan.

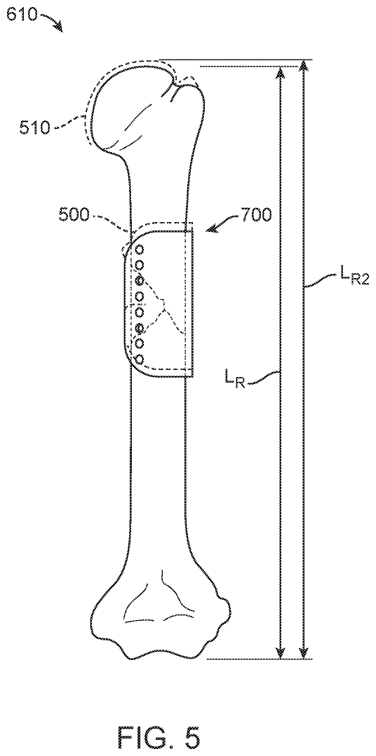

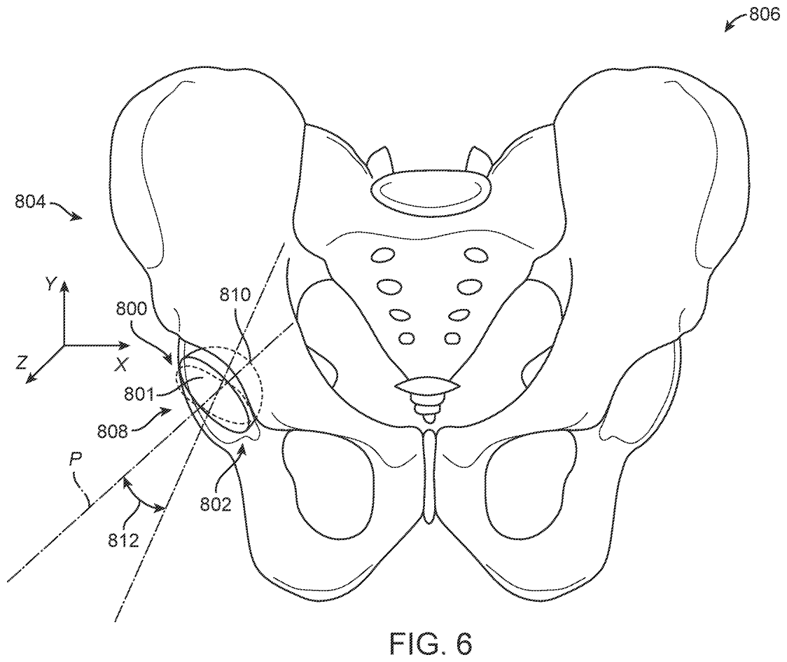

Optionally, at block 118 postoperative imaging of the patient is performed. Postoperative imaging may include a variety of diagnostic images of the patient, such as CT, MM, and other scans and imaging techniques, such as x-rays. From the diagnostic images a three-dimensional (3-D) model of the patient's bone structure, implants, and prosthetics in their postoperative positions is generated. From such 3-D models, the surgeon may determine the extent of a fracture and/or implant installation location and orientation to be secured to the patient's anatomy during surgery. For example, FIGS. 5 and 6 include, in part, postoperative models or images of the patient's anatomy, along with the installed implants and/or prosthetics.

At block 120, the preoperative planned position and orientation of the implants and/or prosthetics and/or the shape of the patient's anatomy, such as a bone of the patient is compared to the postoperative achieved position and orientation of the implants and/or prosthetics and/or the shape of the patient's anatomy, such as a bone of the patient. The deviation of the achieved result from the planned result may be compared both quantitively and qualitatively. For example, the deviation of the position and orientation of an implant or prosthetic may be determined in one or more degrees of freedom, such as one or more rotational degrees of freedom and one or more translational degrees of freedom. In some embodiments, the position may be determined with respect to six degrees of freedom, including three rotational degrees of freedom and three translational degrees for freedom. In some embodiments, such as fracture reduction, the length, diameter, or other dimension of the reduced bone may be compared to a corresponding dimension of in the preoperative plan. FIGS. 5 and 6 show a comparison between models generated as part of a preoperative plans and models generated from postoperative achieved results.

In some embodiments, 2D imaging may be used in the comparison, for example, 2D imaging may be used in the preoperative plan, for example, when patient specific tools and implants are not used. Similarly 2D imaging may be used for the post-operative scans, without the use of 3D scans. The positions of implants or shapes of anatomy in the 2D images, such as x-rays, may be evaluated to determine the outcome of the surgery. In some embodiments, 2D images may be used without 3D images to determine the deviations between the planned and achieved surgical results such as implant placement, fracture reduction, resection, etc.

In some embodiments, the achieved result may be qualitatively compared to the preoperative plan, such as through a ranking and grading system based on the quantitative results. For example, the ranking or grading may be based on the achieved result matching the preoperative plan within one or more thresholds. For example, a scale of 1-5 may be used for ranking or grading the procedure. As an example, for an acetabular cup, a grade of 5 may be given when the orientation and location of the cup is within a first threshold small threshold form the desired position, such as within 1 degree in each of three rotational degrees of freedom and within 1 mm in each of three translational degrees of freedom, while a grade of 4 may be given when the either rotation or translation is within the first threshold and a the other is within a second larger threshold, such as within 3 degrees and 3 mm of the desired orientation and location. A grade of 3 may be given when both the angle and orientation are beyond the first threshold, but less than the second threshold. Similarly, other additional, greater thresholds may be used. In some embodiments, more than one position and orientation may be considered beneficial or acceptable. In some embodiments, the surgical result may be considered acceptable if it is at the one or more positions and orientations or within the range of acceptable positions and orientations.

In some embodiments, doctor compensation for the procedure may be tied to the grade. For example, a first grade, such as a 5 may entitle the doctor to full payment, and lower grades may result in correspondingly lower payments. In some embodiments, surgical results may be tracked over time. If performance over time is shown to be poor, then the doctor may be dropped from the insurance network. In some embodiments if performance is shown to be poor the doctor may be supervised during future surgical procedures, requested to participate in additional educational opportunities, may have reduced surgical privileges at a hospital, or may have their privileges revoked.

As discussed above, FIG. 3 shows the system 310 for carrying out the methods of FIGS. 1 and 2 according to some aspects of the present disclosure. The computing system 312 may include instructions in the form of computer software for automatically generating images of implants in final installation positions on the bone structure images. In some aspects, a surgeon input information into the input device 318 for creating or altering jig images or implant images for a particular patient based on the surgeon's understanding of the particular bone structure of the patient as displayed on the display device 316 during preoperative planning.

FIGS. 3A and 3B depict a healthy left humerus 600 and a fractured right humerus 610, respectively. The healthy left humerus 600 includes a shaft or body 606 extending between a proximal end 602, including the head, and a distal end 604. The length of the healthy left humerus 600, as measured between the proximal end 602 and distal end 604 is LN.

The right fractured humerus 610 includes a shaft or body 616 extending between a proximal end 612, including the head, and a distal end 614. The length of the healthy left humerus 610, as measured between the proximal end 612 and distal end 614 is LF. The fractured bone 610 includes three pieces, a proximal portion 630, a distal portion 632, and a fragment 618. The fracture depicted in FIG. 3B is a three piece comminuted bone fracture. In other embodiments, other types of fractures may occur. In some embodiments, a fracture may include two pieces or more than three pieces. In some embodiments, the bone surface image of the contralateral unfractured bone image may be used as a model of the bone surface image of the reduced fractured bone such that the doctor reduces the fractured bone surface image such that it matches the unfractured bone image, in some embodiments, the reduced image may match an inverse or mirrored unfractured bone image.

When reducing the bone fracture depicted in FIG. 3B, a doctor attempts to close the gaps in between the pieces of bone 630, 632, and 634 by moving the pieces of bone 630, 632, and 634 into their pre fracture orientations and positions. For example, a doctor may align the fracture surface 624 of bone piece 630 with the fracture surface 626 of bone piece 632 and fracture surface 622 of bone piece 618 and to also align the fracture surface 628 of bone piece 632 with the fracture surface 620 of bone piece 618 such that the respective surfaces are mated with each other. For example, surface 624 is mated with fracture surface 626 and fracture surface 622 while fracture surface 628 is mated with the fracture surface 620.

An improperly reduced bone fracture may result in one extremity, such as an arm or leg, being longer or shorter than the opposite extremity. In some embodiments, an improperly reduced bone fracture may result in malrotation rotation or improper angulation. For example, a fractured left femur may be reduced and heal such that the healed left femur is longer than the health right femur. Such differences in lengths can cause problems in patients because one leg may receive greater loads when walking and other parts of the body may adjust to compensate for the different lengths, causing pelvis and back problems. Therefore, a doctor may measure the length of a corresponding healthy bone and use that length to aid in properly reducing the fractured bone.

In some embodiments, some portions of a fractured bone may not be useable when reducing the fracture. For example, pieces of the bone may be missing, too small, or too damaged such that the doctor cannot put them in their proper place when reducing the bone fracture. This may lead to the bone being reduced such that its length is not correct or with bone pieces in an incorrect position or orientation. In some embodiments, angulated bones may produce functional problems and or cosmetic deformities such as excessive varus or valgus angulation, procurvatum, or recurvatum angulation.

FIGS. 4A and 4B depict a reduced preoperative image of a fractured bone 610 and a bone reduction tool 700.

The bone reduction tool 700 has several features and uses, including aiding in reducing the bone fracture and in fixing the fractured bone. For example, during the fracture reduction process the bone portions of the fractured bone 630, 618, and 632 are repositioned such that the fracture surfaces of the respective portions of fractured bone 630, 618, and 632 mate with each other and the bone 610 is reduced to a pre-fracture configuration. In the preoperative planning stages of the surgery, a reduced bone image is formed based on the surface images of the portions of the fractured bone 630, 618, and 632 and other factors, such as the unfractured length of the bone or an unfractured length of a corresponding bone of a patient.

When the bone images are reduced, the bone image may have a unique surface shape. The bone reduction tool 700 includes a bone facing surface 704 that includes one or more portions that are shaped to match respective portions of unique surface shape of the reduced bone. For example, as shown in FIGS. 4A and 4B the bone facing surface 704 of the body of the bone reduction tool 700 spans the fracture such that is makes contact with each of the portions of the fractured bone 630, 618, and 632.

The bone facing surface 704 is an anatomic alignment surface shaped to match the surface of the reduced fractured bone 610 in a single position and orientation. The shape and contours of the bone facing surface 704 may be determined based upon the 3-D modeling images of the patient, a combination of two-dimensional radiographic images of the patient, or a combination of three-dimensional and two-dimensional images of a patient. The shape of the bone facing surface 704 is sometimes referred to as a negative of the anatomic structure with which the bone facing surface 704 aligns or engages. It is a negative because, for example, a protrusion on the anatomic surface structure of the bone surface image of the bone 610 corresponds to a depression on bone facing surface 704 while a depression on the anatomic surface structure of the bone surface image of the bone 610 corresponds to a protrusion on the bone facing surface 704.

A doctor may use a bone reduction tool, such as the bone reduction tool 700, during a fracture repair. For example, after the patient's bone fracture, the doctor may attempt to place the bone reduction tool 700 in its preoperatively planned installation position and orientation. When placing the bone reduction tool 700, the doctor attempts to align or engage the patient specific surface 704 with the anatomic structure of the patient and then observe the alignment or misalignment of the patient specific surface 704 with the portions of fractured bone 618, 630, and 632. The alignment or misalignment of the prosthetic alignment surfaces with patient specific surface 704 with the portions of fractured bone 618, 630, and 632 indicates information to the doctor regarding the reduction of the bone fracture. For example, if the patient specific surface 704 aligns with the surface of the portions of fractured bone 618, 630, and 632, then a doctor may know that the bone 601 has been properly reduced, while misaligned alignment of the surfaces may indicate how the position or orientation of one or more of the portions of fractured bone 618, 630, and 632 should be changed to reduce the bone into the final pre-operatively planned reduced position.

The bone reduction tool 700 may also include apertures 702 that extend between the bone facing surface 704 and the outward facing surface 706. The apertures 702 may be pilot hole guide apertures. As pilot hole guide apertures, the apertures 702 aid in the drilling of pilot holes for use in affixing the bone reduction tool to the bone of the patient. For example, after the bone 610 is reduced, bone reduction tool 700 may be affixed to the portions of fractured bone 618, 630, and 632 and implanted to hold the portions of fractured bone 618, 630, and 632 in place while the bone 610 heals. Fasteners, such as screws may be used to affix the bone reduction tool 700 to the bone 610. By drilling the pilot holes into the portions of fractured bone 618, 630, and 632 while the bone reduction tool 700 holds the portions of fractured bone 618, 630, and 632 in place, the doctor may ensure that the pilot holes are in the proper location.

In some embodiments, the apertures 702 are shaped to receive fasteners, such as screws for affixing the bone reduction tool 700 to the reduced fractured bone of the patient. In such an embodiment, the bone reduction tool 700 is also an implant that holds the portions of fractured bone 618, 630, and 632 in place while the bone heals.

FIG. 5 shows a preoperative image of a bone 610 and implant 700 superimposed with a postoperative image of a bone 510 and implant 500. In some embodiments the images may be three-dimensional models generated using imaging and treatment planning methods as described herein. As shown in FIG. 5, the postoperative bone has a length L_{R2} that is longer than the preoperatively planned length L_{R}. In addition, postoperative position of the implant 500 is displaced from the preoperatively planned position of the implant 700. The images may be analyzed to determine the difference between the desired and actual achieved positions and lengths of implants and anatomy, such as bones. In some embodiments, the proper size of implants, screw or pin lengths, positions and trajectories and/or other parameters may be evaluated. Based on this analysis the success of the procedure may be ranked quantitatively or qualitatively as discussed herein.

FIG. 6 show an image of an acetabular component or implant 801 oriented in an acetabulum 802 of a coxal bone 804 of a pelvic bone 806. The acetabular component 801 is positioned according to preoperatively planned installation position 808, which is in part determined by a prescribed anteversion angle and a prescribed inclination angle of the acetabular component 801. FIG. 6 shows a front view of the pelvic bone 806 and the acetabular component 801 positioned in the acetabulum 802 of the patient's right coxal bone 804. The preoperatively planned central axis P of the acetabular component 801 is also shown. FIG. 6 also shows the postoperative image of the implant 800 superimposed or overlaid with the preoperative image. In some embodiments the images may be three-dimensional models generated using imaging and treatment planning methods as described herein. The acetabular component 801 may also be placed at a location in 810. The location 810 may correspond to the center of the acetabular component 801 or the location of another part of the acetabular component 801. In some embodiments the location 810 may correspond to a depth of the acetabular component 801. The location may be based on the center of mass or center of volume of the implant or based on another reference feature, such as a screw location or an edge location, such as an edge of the implant. The location may be a one, two, or three dimensional location and may be defined in one, two, or three dimensions. As shown in FIG. 6, the postoperative orientation of the central axis of the acetabular component 801 is displaced from the preoperatively planned position of the orientation of the central axis P by an angle 812. As also shown in FIG. 6, the depth of the acetabular component does correspond to the preoperatively planned depth. The images may be analyzed to determine the difference between the desired and actual achieved position and orientation of the acetabular component 801. In some embodiments, the planned leg lengths, center of rotation of the leg and/or the implant and other clinically relevant variables may also be compared to the actual achieved results. Based on this analysis the success of the procedure may be ranked quantitatively or qualitatively as discussed herein.

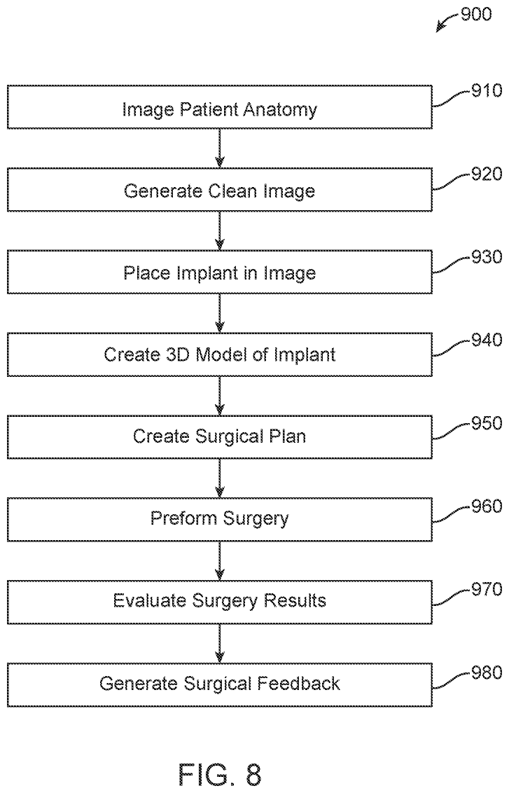

FIG. 8 depicts a digital image of a spinal fusion implant. In some situations where the vertebrae are damaged and can no longer move properly, such as with arthritis, fractures of a vertebra, or as a follow-up surgery for a patient with a severely herniated disc, spinal fusion may be desired. While these procedures may or may not include removal of a part of the vertebra or disc, fusion typically includes installing a graft that attempts to mimic the normal healing process of bones by integrating with sequential vertebra as the bone heals, resulting in two or more vertebra permanently joined together to move as one. The grafts may include a fusion cage, bone autograft from the patient's body, a bone allograft from cadaver bone, or a synthetic graft produced with synthetic materials. After placing the graft, the graft is then secured with implants that will vary based on the location of the procedure, such as standard plates and screws placed from the front of the spine, pedicle screws placed from the rear of the spine, rods through screws placed from the rear of the spine, or fusion cages placed from the front or the side of the spine. Either standard sized or patient specific implants may be used. In some embodiments, the proper size of implants, screw or pin lengths, positions, and trajectories and/or other parameters may be evaluated based on their planned and actual achieved results.

Yet other patients may be treated with a full disc removal and replacement if the issues with that patient's disc or discs are more serious than a herniation. A disc replacement involves replacing the entire disc with a prosthesis designed to imitate the functions of a normal disc, namely carry load and allow for a proper range of motion. The surgeon first removes the disc and then, in some instances, locates additional areas to be removed in the disc cavity to provide for grooves or channels or other features that allow for installation of the prosthesis. The disc replacement prosthetic may include two plates with a compressible, plastic-like piece between the plates. The prosthesis may be installed and secured by attaching one plate to the vertebra above the disc being replaced and the other to the vertebra below, with the surfaces of the plates that attach to the vertebrae having tines or notches that slide into channels cut or drilled onto the surface of the vertebrae facing the prosthesis. As such, these prosthetic devices are secured to the vertebrae and allow a full range of motion in the disc cavity, similar to a healthy spine. The plates may be standard sized, generic plates, or patient specific plates. In some embodiments, the actual achieved disc size and location, such as it's proper placement, may be evaluated and scored based on a comparison with the respective planned parameter.

Finally, circumstances may arise that call for a correction to a deformity in a patient's spine, such as with an osteotomy, which is a procedure where a doctor removes bone of the patient and forces the spine closed with hardware in a new orientation in order to fix the deformity. While there are several types of osteotomies, one example procedure is the Smith-Petersen Osteotomy, which includes removing a wedge of spinal structure from the rear section of the spine and then lengthening the anterior portion to force the wedge closed with hardware. In some circumstances, the discs may be intentionally ruptured and new material added to the front of the spine to assist with the mobility of the spine after forcing the rear side of the spine closed. In some embodiments, the actual achieved correction of a deformity, sizing and placement of implants, and other parameters or variables may be evaluated with respect to the respective planned results.

Each of these four categories of operations can be aided by computer generated data derived from CT, MM, or other scans, such as X-rays. Using these imaging sources, surgeons can more effectively determine the area to be removed from a patient's spine and the proper alignment and positioning for installation of any implants through 3-D modeling and rendering. Based on these 3-D models, some doctors use lasers or peripheral guide pins during medical procedures in an attempt to measure the adequacy of tissue removal or to guide the hardware; however, devices in the art are relatively complex and do not carry a high level of accuracy, which increases the chances of complications for a patient in complex surgeries like spinal procedures.

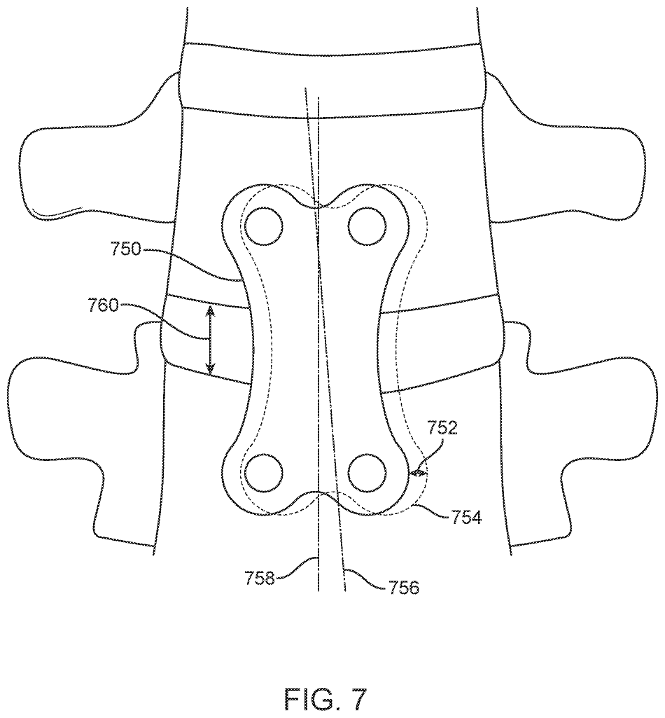

FIG. 7 show a post operative image of an implant 750 attached to the spine of a patient. The implant 750 is positioned as installed during surgery. The preoperative planned implant image 754 is located according to preoperatively planned installation position, which is in part determined by a location and orientation of the implant 750. The orientation of the implant 750 is determined based on a longitudinal axis 758 that extends parallel to the longest dimension of the implant. FIG. 7 shows a rear view of the spine and the implant 750 positioned on the spine. The preoperatively planned longitudinal axis 756 is also shown. In some embodiments, preoperatively planned longitudinal axis 756 may be parallel to the spine. FIG. 7 shows the postoperative image of the implant 750 superimposed or overlaid with the preoperative image of the implant 754. In some embodiments, the images may be three-dimensional models generated using imaging and treatment planning methods as described herein. In some embodiments, the images may be two dimensional images generated using imaging and treatment planning methods as described herein. The location may be a one, two, or three-dimensional location and may be defined in one, two, or three dimensions. For example, when using 2D post operative imaging, then one or two-dimensional locations can be used. When using 3D post operative imaging, then one, two, or three-dimensional imaging may be used. The location may be based on the center of mass or center of volume of the implant or based on another reference feature, such as a screw location or an edge location, such as an edge of the implant.

As shown in FIG. 7, the postoperative orientation of the longitudinal axis 758 of the implant 750 is displaced from the preoperatively planned position of the orientation of the longitudinal axis 756 by an angle. As also shown in FIG. 7, the edges of the implant 750 do not correspond to the preoperatively planned locations of the edges. For example, the lower right edges are displaced by a distance 752. The images may be analyzed to determine the difference between the desired and actual position and orientation of the implant 750. Based on this analysis the success of the procedure may be ranked quantitatively or qualitatively as discussed herein. Based on this analysis, the success of the procedure may be ranked quantitatively or qualitatively as discussed herein.

In FIG. 8, a method 900, according to an embodiment, may start at block 910. At block 102, an anatomy imaging machine generates an image or images of the patent's anatomy from two or three-dimensional image data of the anatomical structure of a patient, for example, as shown in FIGS. 3A, 3B, 5, 6, and 7. The anatomy may be hard tissue, such as bone, or soft tissue, such as organs, muscles, etc. At block 920, the image or images of the patient's anatomy are cleaned by removing noise, inaccurate data, etc. In some embodiments, the images may be cleaned using manual imagine processing and cleaning techniques. In some embodiments, the image may be cleaned using automated techniques, such as through Machine Learning or Artificial Intelligence algorithms.

At block 930, the preoperative planned location of the implant or the patient's anatomy is determined. In some embodiments, a virtual implant image is placed on the image of the patient's anatomy. The image may be the cleaned image or an image that is not cleaned. The pre-operatively planned position represents the desired surgical outcome of the implant and patient's anatomy. In some embodiments, the image may be of an acetabular cup, pelvis, knee, spine or other bone or tissue. In some embodiments, the image may be manipulated to generate preoperatively planned resection models that depict the patient's anatomy after resection or other material removal procedures, such as those depicted in FIGS. 5, 6, 7, 9A, 9B, and 10 and described herein.

In some embodiments, such as to evaluate surgeries or parts of surgical procedures wherein an implant is not used, the image of the patient's anatomy may be modified according to the desired post-operative surgical results. The image may be the cleaned image or an image that is not cleaned. The pre-operatively planned post operative results may include the desired surgical outcome of the resection, material removal, or other modification to a patient's anatomy. In some embodiments, the image may be of an acetabular cup, pelvis, knee, spine or other bone or tissue. In some embodiments, the image may be manipulated to generate preoperatively planned resection models that depict the patient's anatomy after resection or other material removal procedures, such as those depicted in FIGS. 5, 6, 7, 9A, 9B, and 10 and described herein.

At block 940, a 3D model of the implant is created or otherwise generated. The actions at block 940 may occur before or after the actions of block 930. The 3D model of the implant may be a virtual 3D model representing the size and shape of the physical implant. The virtual implant may be placed at a position and orientation with respect to the patient's anatomy that corresponds or otherwise represented the planned location of the physical implant on the patient's physical anatomy during or after surgery.

A patient-specific device generator may generate an implant image superimposed in an installation position on the image of the patient's anatomy. For example, FIGS. 4A, 5, 6 and 7 include aspects of the preoperative implant image at the installation position. The implant image is positioned in its final, implanted position and orientation, regardless of the state of the patient's anatomy in the image of the patient's anatomy. The implant image may be a patient specific implant image, having a surface that is shaped to match or otherwise mate with the bone surface image of the patient. In some embodiments, the implant may be a standard size or non-patient specific implant. An actualized or physical patient specific implant formed based on the implant image may have an physical surface that matches or otherwise mates with the reduced fractured bone of the patient. Similarly, implant images may be generated that depict the preoperative planned position of one or more prosthetics or other implants, such as an acetabular cup, spine fusion implant, knee replacement implants, etc. that are positioned with respect to the patient's anatomy in a desired optimal position or range of acceptable positions.

Similarly spinal implant images may be generated that represent the preoperatively planned position of a spine implant, such as those used during decompression, spinal fusion, disc replacement or spinal corrective osteotomy placed. In some embodiments, the implant images may show a range of acceptable positions, an ideal size of the implant, ideal screw lengths, sizes, and trajectories.

At block 950 a surgical plan is created. The surgical or preoperative plan may include the steps and tools for performing the surgery including incision locations, resection locations and amounts of anatomy, such as bone and/or soft tissue to be removed, implants to be used, tools to be used for resection, incision, and implant placement and affixment, and etc.

At block 960, a doctor may perform the orthopedic surgery and install an implant, reduce a fracture, or perform other orthopedic procedures as detailed in the surgical or preoperative plan.

At block 970 the surgical results are evaluated. Postoperative imaging of the patient may be performed. Postoperative imaging may include a variety of diagnostic images of the patient, such as CT, MM, and other scans and imaging techniques, such as x-rays, with or without the use of artificial intelligence algorithms, such as neural networks trained based on imaging of past surgeries, such as annotated images of past surgeries and their planning images with annotations for expected and actual results for the parameters and/or scoring. From the diagnostic images, a three-dimensional (3-D) model of the patient's bone structure, implants, and/or prosthetics in their postoperative positions is generated. From such 3-D models, the surgeon may determine the extent of a fracture and/or implant installation location and orientation that was secured to the patient's anatomy during surgery or the modifications to the patient's anatomy during surgery. For example, FIGS. 5, 6, 7, 9A, 9B, and 10 include, in part, postoperative models or images of the patient's anatomy, along with the installed implants and/or prosthetics.

The preoperative planned position and orientation of the implants and/or prosthetics and/or the shape of the patient's anatomy, such as a bone of the patient is compared to the postoperative achieved position and orientation of the implants and/or prosthetics and/or the shape of the patient's anatomy, such as a bone of the patient, with or without the use of artificial intelligence algorithms, such as neural networks trained based on imaging of past surgeries, such as annotated images of past surgeries and their planning images with annotations for expected and actual results for the parameters and/or scoring. The deviation of the achieved result from the planned result may be compared both quantitively and qualitatively. For example, the deviation of the position and orientation of an implant or prosthetic may be determined in one or more degrees of freedom or along one or more axis, such as one or more rotational degrees of freedom and one or more translational degrees of freedom. In some embodiments, the position may be determined with respect to six degrees of freedom, including three rotational degrees of freedom and three translational degrees for freedom, based on manual indication of the differences or automated analysis of the differences, such as with image processing software. FIGS. 5, 6, and 7 show a comparison between models generated as part of a preoperative plans and models generated from postoperative achieved results.

In some embodiments, 2D imaging may be used in the comparison, for example, 2D imaging may be used in the preoperative plan, for example, when patient specific tools and implants are not used. Similarly 2D imaging may be used for the post-operative scans, without the use of 3D scans. The positions of implants or shapes of anatomy in the 2D images, such as x-rays, may be evaluated to determine the outcome of the surgery. In some embodiments, 2D images may be used without 3D images to determine the deviations between the planned and achieved surgical results such as implant placement, fracture reduction, resection, etc, based on manual indication of the differences or automated analysis of the differences, such as with image processing software. In some embodiments, artificial intelligence algorithms trained with images, as discussed herein may be used in the determination.

In some embodiments, the achieved result may be qualitatively compared to the preoperative plan, such as through a ranking and grading system based on the quantitative results. For example, the ranking or grading may be based on the achieved result matching the preoperative plan within one or more thresholds. For example, a scale of 1-5 may be used for ranking or grading the procedure. As an example, for an acetabular cup, a grade of 5 may be given when the orientation and location of the cup is within a first threshold small threshold form the desired position, such as within 1 degree in each of three rotational degrees of freedom and within 1 mm in each of three translational degrees of freedom, while a grade of 4 may be given when the either rotation or translation is within the first threshold and a the other is within a second larger threshold, such as within 3 degrees and 3 mm of the desired orientation and location. A grade of 3 may be given when both the angle and orientation are beyond the first threshold, but less than the second threshold. Similarly, other additional, greater thresholds may be used. In some embodiments, more than one position and orientation may be considered beneficial or acceptable. In some embodiments, the surgical result may be considered acceptable if it is at the one or more positions and orientations or within the range of acceptable positions and orientations.

In some embodiments, doctor compensation for the procedure may be tied to the grade. For example, a first grade, such as a 5 may entitle the doctor to full payment, and lower grades may result in correspondingly lower payments. In some embodiments, surgical results may be tracked over time. If performance over time is shown to be poor, then the doctor may be dropped from the insurance network. In some embodiments if performance is shown to be poor the doctor may be supervised during future surgical procedures, requested to participate in additional educational opportunities, may have produced surgical privileges at a hospital, or may have their privileges revoked.

In some embodiments, data or the results of the analysis, such as a scorecard, may be provided to patients to use to find high quality and/or low-cost surgeons. In some embodiments, data may be used by hospitals or surgery centers to promote high performing surgeons and/or to require additional training and/or to restrict or remove privileges for low performing surgeons. In some embodiments, insurers may direct patients to high performing and/or low cost providers and/or to increase or decrease hospital and/or surgeon reimbursement based upon review of pre and post procedure analysis. In some embodiments, the data and/or results may be used for any combination, including all of the above.

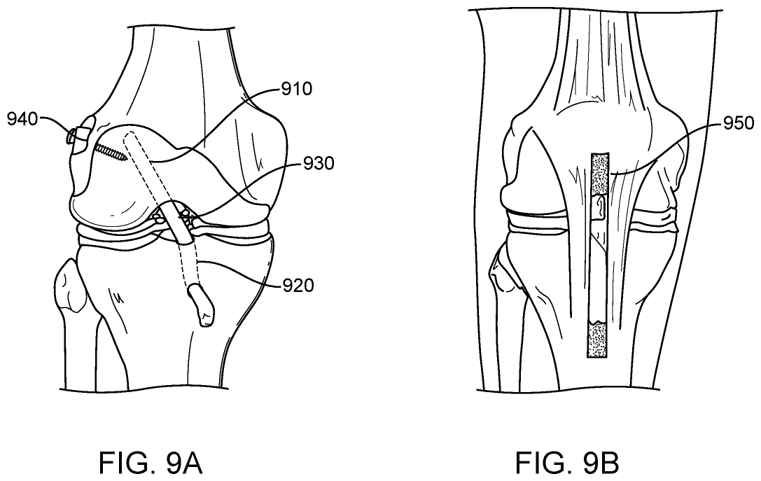

FIGS. 9A and 9B depict aspects of an anterior cruciate ligament (ACL) reconstruction according to one or more embodiments disclosed herein. In ACL reconstruction, preoperative planned position and orientation of the implants and/or prosthetics and/or the shape of the patient's anatomy, such as a bone of the patient is compared to the postoperative achieved position and orientation of the implants and/or prosthetics and/or the shape of the patient's anatomy, such as a bone of the patient. The deviation of the achieved result from the planned result may be compared both quantitively and qualitatively. For example, the deviation of the positions and orientations of the femoral tunnel 910 and the tibial tunnel 920 in the postoperative position may be evaluated with respect to their preoperatively planned positions and orientations. Positions and deviations may be expressed in three dimensions, such as in the anterior-posterior or sagittal planes, medial-lateral or coronal planes, and the proximal-distal or transverse planes. In some embodiments, the length and width of the soft tissue graft, such as the patellar tendon-bone graft 950 as measured from and end of the incision at the patella to an end of the incision at the tibia. The width may be measured in a medial-lateral direction or axis from a first side of the incision to a second side of the incision. Similarly, a soft tissue graft from the hamstrings or quadriceps may be measured in both length and width and may be compared to a pre-operatively planned length and width. The post-surgical alignment of the femur and tibia may be quantified in anterior translation of the tibia relative to the femur during normal range of motion of the knee. In some embodiments, the location and orientation of the placement of the interference screws 940 in the femoral and tibial bone tunnels may also be analyzed.

In some embodiments, 2D imaging may be used in the comparison or analysis, for example, 2D imaging may be used in the preoperative plan, for example, when patient specific tools and implants are not used. Similarly 2D imaging may be used for the post-operative scans, without the use of 3D scans. The positions of implants or shapes of anatomy in the 2D images, such as x-rays, may be evaluated to determine the outcome of the surgery. In some embodiments, 2D images may be used without 3D images to determine the deviations between the planned and achieved surgical results such as implant placement, fracture reduction, resection, the length and width of the soft tissue graft, such as the patellar tendon-bone graft 950 as measured from and end of the incision at the patella to an end of the incision at the tibia, etc.

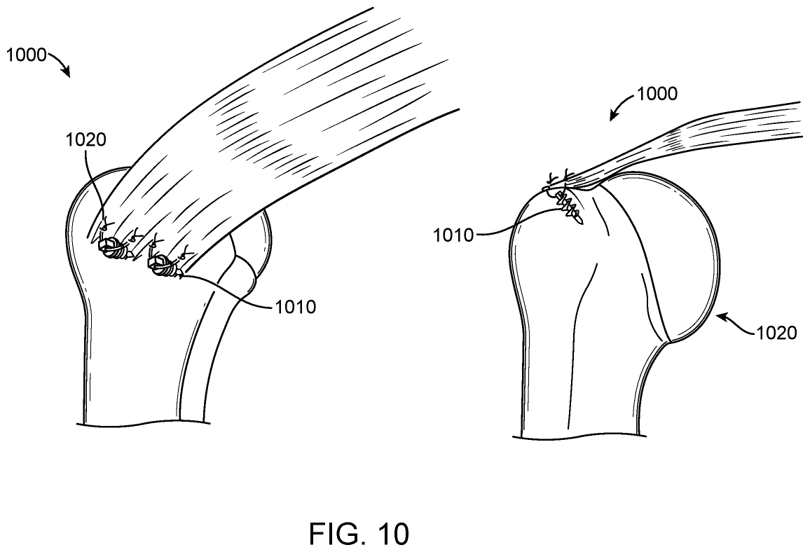

FIG. 10 shows aspects of a rotor cuff 1000 repair. In rotor cuff repair, preoperative planned position and orientation of the implants, sutures, and/or prosthetics and/or the shape of the patient's anatomy, such as a bone of the patient is compared to the postoperative achieved position and orientation of the implants and/or prosthetics and/or the shape of the patient's anatomy, such as a bone of the patient. The deviation of the achieved result from the planned result may be compared both quantitively and qualitatively. For example, the deviation of the positions and orientations of the suture anchors 1010 and sutures 1020 in the postoperative position may be evaluated with respect to their preoperatively planned positions and orientations. Suture locations in the supraspinatus and infraspinatus may be evaluated. Positions and deviations may be expressed in three dimensions, such as in the anterior-posterior or sagittal planes, medial-lateral or coronal planes, and the proximal-distal or transverse planes. In some embodiments, the restoration of anatomic glenohumeral relationship may be analyzed. For example, the amount or distance of superior migration and anterior or posterior subluxation of the humerus relative to the glenoid as compared to normal rotator cuff function may also be analyzed.

In some embodiments, 2D imaging may be used in the comparison or analysis, for example, 2D imaging may be used in the preoperative plan, for example, when patient specific tools and implants are not used. Similarly 2D imaging may be used for the post-operative scans, without the use of 3D scans. The positions of implants or shapes of anatomy in the 2D images, such as x-rays, may be evaluated to determine the outcome of the surgery. In some embodiments, 2D images may be used without 3D images to determine the deviations between the planned and achieved surgical results such as implant placement, fracture reduction, resection, etc.

The various embodiments described above can be combined to provide further embodiments. Aspects of the embodiments can be modified, if necessary to employ concepts of the various patents, applications and publications to provide yet further embodiments.

The present disclosure includes the following numbered clauses.

Clause 1. A surgical planning and evaluation system comprising: a processor; and memory comprising instructions that when executed by the processors cause the system to: receive an image of a patient's anatomy before a surgery; generate a surgical plan for performing the surgery, the surgical plan including a planned surgical result; receive an image of the patient's anatomy after surgery, the image comprising data representing the achieved surgical result; digitally compare the planned surgical result with the achieved surgical result; and generate a quantification the surgical result based on the digital comparison.

Clause 2. The surgical planning and evaluation system of claim 1, wherein: the quantification of the surgical result is a percent of planned surgical result achieved.

Clause 3. The surgical planning and evaluation system of claim 1, wherein the memory further comprises instructions that when executed by the processors, further cause the system to: compare the quantification of the surgical result to a threshold, and generate feedback indicating a successful surgery if the quantification of the surgical result is less the threshold.

Clause 4. The surgical planning and evaluation system of claim 3, wherein: the quantification includes a spatial difference between a location of a digital implant in the surgical plan and a location of a digital representation of a physical implant in the image of the patient's anatomy after surgery.

Clause 5. The surgical planning and evaluation system of claim 1, wherein: the surgical plan including the planned surgical result includes a digital representation of the planned surgical result.

Clause 6. The surgical planning and evaluation system of claim 5, wherein: the digital representation of the planned surgical result includes a digital representation of an implant in a planned post-operative position.

Clause 7. The surgical planning and evaluation system of claim 1, wherein the memory further comprises instructions that when executed by the processors, further cause the system to: compare the quantification of the surgical result to a threshold, and generate feedback indicating a successful surgery based on the quantification of the surgical result.

Clause 8. The surgical planning and evaluation system of claim 1, wherein: the digital representation of the planned surgical result includes a digital representation of an implant in a planned post-operative position, data representing the achieved surgical result surgical result includes a digital representation of a physical implant in an actual post-operative position, and wherein the memory further comprises instructions that when executed by the processors, further cause the system to: compare the quantification of the surgical result to a threshold, and generate feedback indicating a successful surgery based on the quantification of the surgical result.

Clause 9. The surgical planning and evaluation system of claim 8, wherein: the quantification includes a spatial difference between a location of the digital representation of the implant in the planned post-operative position and the location of the digital representation of the physical implant in the actual post-operative position.

Clause 10. The surgical planning and evaluation system of claim 9, wherein the implant is a acetabular cup, a spinal fusion implant, bone reduction tool, or a screw.

Clause 11. A surgical planning and evaluation method comprising: receiving an image of a patient's anatomy before a surgery; generating a surgical plan for performing the surgery, the surgical plan including a planned surgical result; receiving an image of the patient's anatomy after surgery, the image comprising data representing the achieved surgical result; digitally comparing the planned surgical result with the achieved surgical result; and generating a quantification the surgical result based on the digital comparison.

Clause 12. The surgical planning and evaluation method of claim 11, wherein: the quantification of the surgical result is a percent of planned surgical result achieved.

Clause 13. The surgical planning and evaluation method of claim 11, further comprising: comparing the quantification of the surgical result to a threshold, and generating feedback indicating a successful surgery if the quantification of the surgical result is less the threshold.

Clause 14. The surgical planning and evaluation method of claim 13, wherein: the quantification includes a spatial difference between a location of a digital implant in the surgical plan and a location of a digital representation of a physical implant in the image of the patient's anatomy after surgery.

Clause 15. The surgical planning and evaluation method of claim 11, wherein: the surgical plan including the planned surgical result includes a digital representation of the planned surgical result.

Clause 16. The surgical planning and evaluation method of claim 15, wherein: the digital representation of the planned surgical result includes a digital representation of an implant in a planned post-operative position.

Clause 17. The surgical planning and evaluation method of claim 11, further comprising: comparing the quantification of the surgical result to a threshold, and generating feedback indicating a successful surgery based on the quantification of the surgical result.

Clause 18. The surgical planning and evaluation method of claim 11, wherein: the digital representation of the planned surgical result includes a digital representation of an implant in a planned post-operative position, data representing the achieved surgical result surgical result includes a digital representation of a physical implant in an actual post-operative position, and further comprising: comparing the quantification of the surgical result to a threshold; and generating feedback indicating a successful surgery based on the quantification of the surgical result.

Clause 19. The surgical planning and evaluation method of claim 18, wherein: the quantification includes a spatial difference between a location of the digital representation of the implant in the planned post-operative position and the location of the digital representation of the physical implant in the actual post-operative position.

Clause 20. The surgical planning and evaluation method of claim 19, wherein the implant is an acetabular cup, a spinal fusion implant, bone reduction tool, or a screw.

Clause 21. The surgical planning and evaluation method or system of any one of claims 1-20, wherein the surgical plan for performing the surgery includes a surgical plan for placing a hip replacement implant, a knee replacement implant, a spinal surgery implant, a fracture procedure implant, or sports medicine replacement implant.

Clause 22. The surgical planning and evaluation method or system of any one of claims 1-21, wherein the generating a quantification of the surgical result includes using an artificial intelligence algorithm trained using image of past surgical results and receiving as input the image of the patient's anatomy after surgery to quantify the surgical result.

Clause 23. The surgical planning and evaluation method or system of any one of claims 1-22, further comprising receiving digital data from a medical professional regarding the surgical result and wherein generating a quantification the surgical result based on the digital comparison and the received digital data from the medical professional.

Clause 24. The surgical planning and evaluation method or system of any one of claims 1-23, further comprising outputting deficiencies in the surgical result based on the comparison.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A surgical planning and evaluation system comprising:
a processor; and
memory comprising instructions that when executed by the processors cause the system to:
receive an image of a patient's anatomy before a surgery;
generate a surgical plan for performing the surgery, the surgical plan including a planned surgical result;
receive an image of the patient's anatomy after surgery, the image comprising data representing the achieved surgical result;
digitally compare the planned surgical result with the achieved surgical result; and
generate a quantification the surgical result based on the digital comparison.

2. The surgical planning and evaluation system of claim 1, wherein:
the quantification of the surgical result is a percent of planned surgical result achieved.

3. The surgical planning and evaluation system of claim 1, wherein the memory further comprises instructions that when executed by the processors, further cause the system to:
compare the quantification of the surgical result to a threshold, and
generate feedback indicating a successful surgery if the quantification of the surgical result is less the threshold.

4. The surgical planning and evaluation system of claim 3, wherein:
the quantification includes a spatial difference between a location of a digital implant in the surgical plan and a location of a digital representation of a physical implant in the image of the patient's anatomy after surgery.

5. The surgical planning and evaluation system of claim 1, wherein:
the surgical plan including the planned surgical result includes a digital representation of the planned surgical result.

6. The surgical planning and evaluation system of claim 5, wherein:
the digital representation of the planned surgical result includes a digital representation of an implant in a planned post-operative position.

7. The surgical planning and evaluation system of claim 1, wherein the memory further comprises instructions that when executed by the processors, further cause the system to:
compare the quantification of the surgical result to a threshold, and
generate feedback indicating a successful surgery based on the quantification of the surgical result.

8. The surgical planning and evaluation system of claim 1, wherein:
the digital representation of the planned surgical result includes a digital representation of an implant in a planned post-operative position,
data representing the achieved surgical result surgical result includes a digital representation of a physical implant in an actual post-operative position, and
wherein the memory further comprises instructions that when executed by the processors, further cause the system to:
compare the quantification of the surgical result to a threshold, and
generate feedback indicating a successful surgery based on the quantification of the surgical result.

9. The surgical planning and evaluation system of claim 8, wherein:
the quantification includes a spatial difference between a location of the digital representation of the implant in the planned post-operative position and the location of the digital representation of the physical implant in the actual post-operative position.

10. The surgical planning and evaluation system of claim 9, wherein the implant is a acetabular cup, a spinal fusion implant, bone reduction tool, or a screw.

11. A surgical planning and evaluation method comprising:
receiving an image of a patient's anatomy before a surgery;
generating a surgical plan for performing the surgery, the surgical plan including a planned surgical result;
receiving an image of the patient's anatomy after surgery, the image comprising data representing the achieved surgical result;
digitally comparing the planned surgical result with the achieved surgical result; and
generating a quantification the surgical result based on the digital comparison.

12. The surgical planning and evaluation method of claim 11, wherein:
the quantification of the surgical result is a percent of planned surgical result achieved.

13. The surgical planning and evaluation method of claim 11, further comprising:
comparing the quantification of the surgical result to a threshold, and
generating feedback indicating a successful surgery if the quantification of the surgical result is less the threshold.

14. The surgical planning and evaluation method of claim 13, wherein:
the quantification includes a spatial difference between a location of a digital implant in the surgical plan and a location of a digital representation of a physical implant in the image of the patient's anatomy after surgery.

15. The surgical planning and evaluation method of claim 11, wherein:
the surgical plan including the planned surgical result includes a digital representation of the planned surgical result.

16. The surgical planning and evaluation method of claim 15, wherein:
the digital representation of the planned surgical result includes a digital representation of an implant in a planned post-operative position.

17. The surgical planning and evaluation method of claim 11, further comprising:
comparing the quantification of the surgical result to a threshold, and
generating feedback indicating a successful surgery based on the quantification of the surgical result.

18. The surgical planning and evaluation method of claim 11, wherein:
the digital representation of the planned surgical result includes a digital representation of an implant in a planned post-operative position,
data representing the achieved surgical result surgical result includes a digital representation of a physical implant in an actual post-operative position, and
further comprising:
comparing the quantification of the surgical result to a threshold; and
generating feedback indicating a successful surgery based on the quantification of the surgical result.

19. The surgical planning and evaluation method of claim 18, wherein:
the quantification includes a spatial difference between a location of the digital representation of the implant in the planned post-operative position and the location of the digital representation of the physical implant in the actual post-operative position.

20. The surgical planning and evaluation method of claim 19, wherein the implant is an acetabular cup, a spinal fusion implant, bone reduction tool, or a screw.
